(19) United States
(12) Patent Application Publication
(10) Pub. No.: US 2002/0090333 A1
(43) Pub. Date: Jul. 11, 2002

(54) Sanitizing device for nursing bottles and teats

(76) Inventor: Yuan-Fu Chang, Feng-Yuan City (TW)

Correspondence Address: CHARLES E. BAXLEY, ESQUIRE, 5th Floor, 59 John Street, New York, NY, 10038, US

(21) Appl. No.: 09/756,602

(22) Filed: Jan. 8, 2001

(51) Int. Cl. A61L 2/00; A61L 9/00; B01B 1/00; B01D 1/00; B01J 2/00

(52) U.S. Cl. 422/300; 422/302; 422/307

(57) ABSTRACT

A sanitizing device includes a first part having a hearing member connected to an inside of a bottom flange and a sanitizing bulb connected to an inside of a top flange on the first part. A rack is connected between two side flanges of the first part. A second part is fixedly connected to the first part and a cover is pivotally connected to the side flanges. A lower edge of the cover is located on a top edge of the second part. A box is removably inserted through the second part and located between two inclined ramps above the heating member.

### FIELD OF THE INVENTION

The present invention relates to a sanitizing device for receiving nursing bottles and teats and a sanitizing bulb is connected to an inside of the device for sanitizing the nursing bottles and teats.

### BACKGROUND OF THE INVENTION

A conventional sanitizing device 10 for sanitizing the nursing bottles is shown in FIG. 1 and generally includes a bowl 11 in which a heating member 14 is connected and a plate 15 is connected on the heating member 14. A rack 13 is located above the plate 15 so that nursing bottles 16 are put on the rack 13. A cover 12 is mounted to the bowl 1 1. Water is put in the plate 15 and heated by the heating member 14 so as to generate vapor which involves high temperature to achieve the sanitize purpose. The vapor is cooled and drained into the plate 15 after the heating member 14 is shut off. However, water drops are attached on the nursing bottles 16 so that the users have to wipe the water drops before using the nursing bottles 16. Besides, the clothes or tissues used to wipe the nursing bottles 16 involve chemical products such as blend agent or fluorescent agent which is not good for infants. Furthermore, the water collected in the plate 15 from cooled vapor becomes a germ-rich spot if the water is not frequently cleaned. The conventional sanitizing device for sanitizing the nursing bottles is designed for the nursing bottles so that other related stuff such bibs, teats and cleaning brushes are not suitable to be processed in the conventional sanitizing device.

### SUMMARY OF THE INVENTION

In accordance with one aspect of the present invention, there is provided a sanitizing device and comprising a first part having a top flange and a bottom flange respectively extending from the first part. Two side flanges are connected between the top flange and the bottom flanges. A hearing member is connected to an inside of the bottom flange and a sanitizing bulb is connected to an inside of the top flange. A rack is connected between the two side flanges. A second part is fixedly connected to the first part and is located connected between the two side flanges. A cover has two arms pivotally connected to the side flanges. A lower edge of the cover is located on a top edge of the second part.

The primary object of the present invention is to provide a sanitizing device which employs sanitizing bulb to sanitize the nursing bottles, the teats, the bib and brushes.

These and further objects, features and advantages of the present invention will become more obvious from the following description when taken in connection with the accompanying drawings which show, for purposes of illustration only, several embodiments in accordance with the present invention.

### BRIEF DESCRIPTION OF THE DRAWINGS

FIG. 1 is a cross sectional view to show a conventional sanitizing device for nursing bottles;

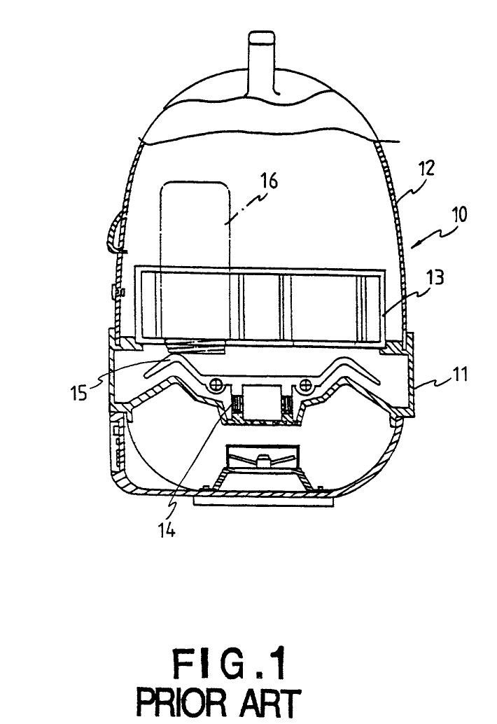

FIG. 2 is an exploded view to show a sanitizing device of the present invention, and

FIG. 3 is a perspective view to show the sanitizing device of the present invention.

### DETAILED DESCRIPTION OF THE PREFERRED EMBODIMENT

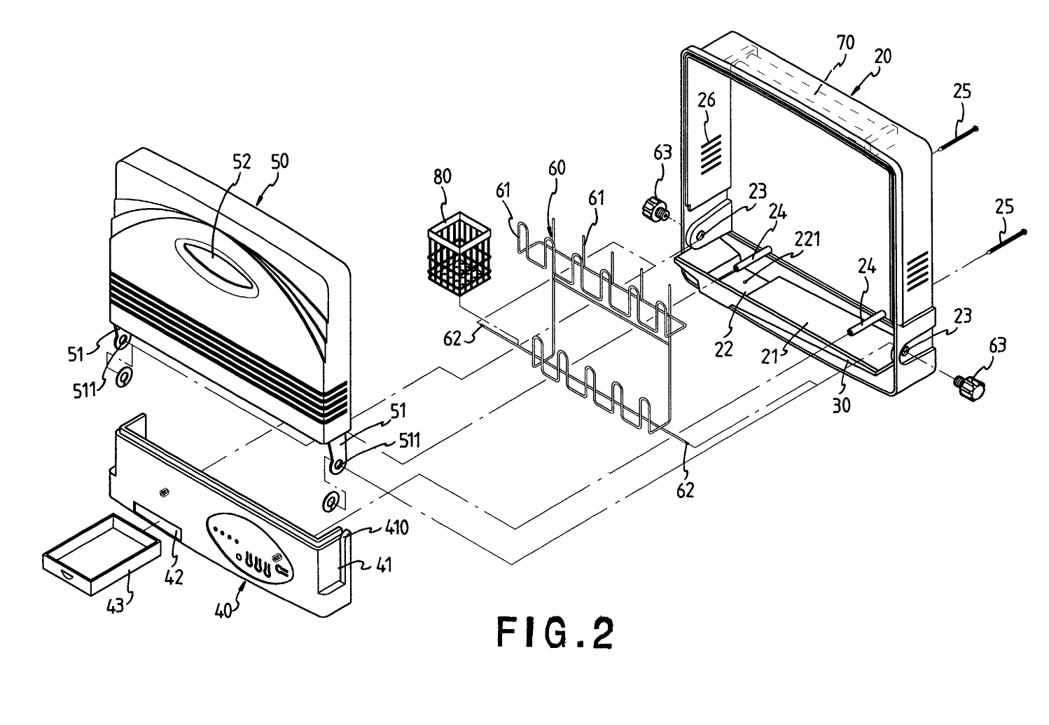

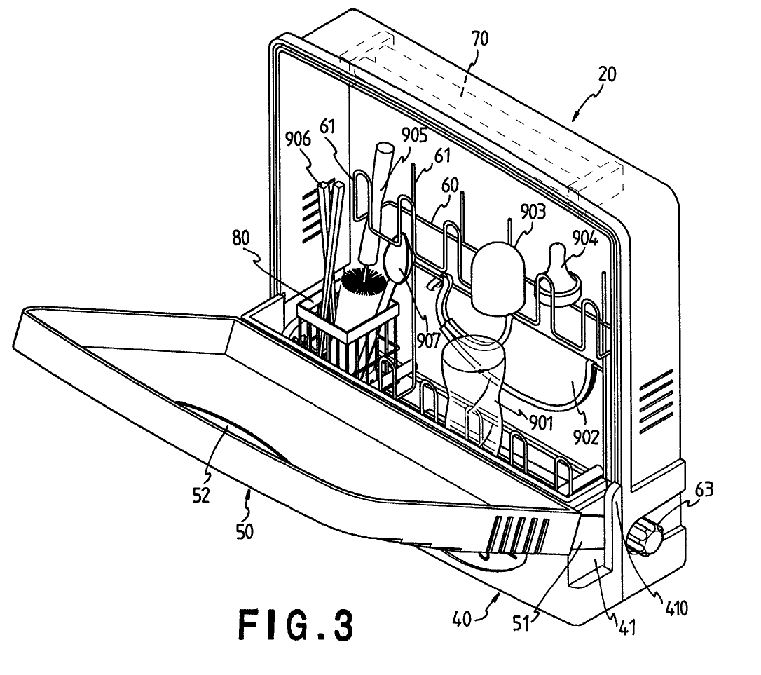

Referring to FIGS. 2 and 3, the sanitizing device of the present invention comprises a first part 20 which has a top flange and a bottom flange respectively extending from the first part 20. Two side flanges extend from two sides of the first part 20 and are connected between the top flange and the bottom flanges. A plurality of ventilation holes 26 are defined through each of the two side flanges of the first part 20 and each side flanges has a hole 23 defined therethrough. A hearing member 30 is connected to an inside of the bottom flange and a sanitizing bulb 70 is connected to an inside of the top flange. Two inclined ramps 21 extend from the two side flanges and a drain gap 221 is located between the two inclined ramps 21. The heating member 30 is located beneath the two inclined ramps 21. Two tubes 24 extend from the first part 20. A rack 60 having two connection ends 62 is connected between the two side flanges and has a plurality of protrusions 61 so that a nursing bottle 901, a bib 902, a teat 904 and a cap 903 for the nursing bottle 901 can be mounted to the protrusions 61 as shown in FIG. 3. A basket 80 is put beside the rack 60 for chopsticks 906, a spoon 907 and a brush 905 put therein.

A second part 40 is fixedly connected to the first part 20 by extending two screws 25 through the first part 20 and the two tubes 24 and being engaged with the second part 40. The second part 40 is connected between the two side flanges. The second part 40 has an operation panel for activating the hearing member 30 and the sanitizing bulb 70. An aperture 42 is defined through the second part 40 and a box 43 is removbaly inserted into the aperture 42 and engaged with the drain gap 221 so as to collect water therein. Two retaining bars 410 respectively extend from two ends of the second part 40 and a slot 41 is defined between each of the two ends of the second part 40 and respective one of the two retaining bars 410.

A cover 50 having two arms 51 which extend through the two slots 41 in the second part and are pivotally connected to the side flanges. A lower edge of the cover 50 is located on a top edge of the second part 40. Each of the arms 51 has a hole 511 and two bolts 63 extend through the two holes 23 in the side flanges of the first part 20 and the holes 511 in the arms 51 and are engaged with the two connection ends 62 of the rack 60. Therefore, the cover 50 can be pivoted about the two bolts 63 to open the sanitizing device as shown in FIG. 2. A window 52 is defined through the cover 50 so as to conveniently observe the interior of the sanitizing device.

The sanitizing device of the present invention can sanitize different shapes or types of nursing items and the design of the cover 50 allows the user to easily put or pick the items in the sanitizing device.

While we have shown and described various embodiments in accordance with the present invention, it should be clear to those skilled in the art that further embodiments may be made without departing from the scope and spirit of the present invention.

What is claimed is:

1. A sanitizing device comprising:
a first part having a top flange and a bottom flange respectively extending from a top edge and a bottom edge of said first part, two side flanges extending from said first part and connected between said top flange and said bottom flanges, a hearing member connected to an inside of said bottom flange and a sanitizing bulb connected to an inside of said top flange, a rack connected between said two side flanges;
a second part fixedly connected to said first part and connected between said two side flanges, and a cover having two arms which are pivotally connected to said side flanges, a lower edge of said cover located on a top edge of said second part.

2. The sanitizing device as claimed in claim 1 further comprising two inclined ramps extending from said two side flanges and a drain gap located between said two inclined ramps, said heating member located beneath said two inclined ramps, a box removably extending through said second part and inserted in said drain gap.

3. The sanitizing device as claimed in claim 1 further comprising two retaining bars respectively extending from two ends of said second part, a slot defined between each of said two ends of said second part and respective one of said two retaining bars, said two arms extending through said two slots and pivotally connected to said two side flanges of said first part.

4. The sanitizing device as claimed in claim 3 further comprising two bolts extending through said two side flanges of said first part and said two arms.

5. The sanitizing device as claimed in claim 1 further comprising a plurality of ventilation holes defined through each of said two side flanges of said first part.

6. The sanitizing device as claimed in claim 1 further comprising two tubes extending from said first part and two screws extending through said first part and inserted into said two tubes and engaged with said second part.

7. The sanitizing device as claimed in claim 1 further comprising a window defined through said cover.
